(19) United States
(12) Patent Application Publication
(10) Pub. No.: US 2018/0267026 A1
(43) Pub. Date: Sep. 20, 2018

(54) METHOD FOR MANUFACTURING A MEASURING KIT AND METHOD FOR ACCELERATING AN ATTACHMENT OF AN ORGANIC MOLECULE TO A CARRIER

(71) Applicant: SIWARD Crystal Technology Co., Ltd., Taichung City (TW)

(72) Inventors: Chuen-Yuan Hsu, Taichung City (TW); Yi-Heui Hsieh, Taichung City (TW); Chiao-Wen Huang, Taichung CIty (TW)

(21) Appl. No.: 15/919,968

(22) Filed: Mar. 13, 2018

(30) Foreign Application Priority Data
Mar. 14, 2017 (TW) 106108408

(51) Int. Cl. G01N 33/531

(52) U.S. Cl. CPC G01N 33/531; G01N 2333/535; G01N 2333/57; G01N 2333/495

(57) ABSTRACT

A method for manufacturing a measuring kit is disclosed. The method for manufacturing a measuring kit includes steps of providing a carrier including a substrate having a surface; coating a silane onto the surface of the substrate; providing an organic molecule; adding the organic molecule onto the carrier; outputting a microwave energy by a coaxial cable; and emitting the microwave energy through a radiator to microwave the carrier and the organic molecule uniformly to coat the organic molecule onto the carrier carrying the silane, wherein the measuring kit is used to apply an enzyme-linked immunosorbent assay.

### CROSS REFERENCE TO RELATED APPLICATIONS

The application claims the benefit of Taiwan Patent Application No. 106108408, filed on Mar. 14, 2017, at the Taiwan Intellectual Property Office, the disclosures of which are incorporated herein in their entirety by reference.

### FIELD OF THE INVENTION

The present invention is related to a method for manufacturing a measuring kit and a method for accelerating an attachment of an organic molecule to a carrier. The methods output a microwave energy by a coaxial cable and emit the microwave energy through a radiator uniformly.

### BACKGROUND OF THE INVENTION

There are a variety of sensing methods on the market. Common sensing methods include enzyme-linked immunosorbent assay (ELISA). ELISA is a widely used sensing method, and it has a multi-year history. There are at least two types of ELISA, wherein in one type of ELISA, the target object is an antigen and in another type, the target object is an antibody. They are discussed as follows.

When the target object is an antigen, ELISA includes the following steps: coating a specific antibody on a plastic plate, wherein the time for coating is about 12-18 hours, and then washing away excess antibodies after the completion of the coating; adding the target object to carry out a reaction with the coated antibody, wherein the reaction time is about 0.5-2 hours, and if the target object contains an antigen reactive with the coated antibody, the antigen will carry out a specific binding with the coated antibody on the plastic plate; after washing away excess target objects, adding an antibody with an enzyme reactive with the antigen to bind with the antigen, wherein the time for binding is about 0.5-1 hours; and then after washing away excess un-bound antibodies with an enzyme, adding a substrate for the enzyme to carry out a color reaction, wherein the time for the color reaction is about 0.5 hour, and then reading the result of the color reaction (i.e. absorbance (OD value)), wherein it takes about 1-2 days to complete the entire test.

When the target object is an antibody, ELISA includes the following steps: coating a known antigen on a plastic plate, wherein the time for coating is about 12-18 hours, and then washing away excess antigens after the completion of the coating; adding the target object to carry out a reaction with the coated antigen, wherein the reaction time is about 0.5-2 hours, and if the target object contains a primary antibody reactive with the coated antigen, the primary antibody will carry out a specific binding with the coated antigen on the plastic plate; after washing away excess target objects, adding a secondary antibody with an enzyme to bind with the primary antibody, wherein the time for binding is about 0.5-2 hours; and then after washing away excess un-bound secondary antibodies with an enzyme, adding a substrate for the enzyme to carry out a color reaction, wherein the time for the color reaction is about 0.5 hour, and then reading the result of the color reaction (i.e. absorbance (OD value)), wherein it takes about 1-2 days to complete the entire test.

Although ELISA is widely used, its operation is time-consuming; especially the coating time is as long as 12-18 hours. In order to improve its efficiency, improving the existing ELISA is an important issue to the skilled person in the art.

### SUMMARY OF THE INVENTION

In accordance with one aspect of the present invention, a method for manufacturing a measuring kit is disclosed. The method for manufacturing a measuring kit includes steps of providing a carrier including a substrate having a surface; coating a silane onto the surface of the substrate; providing an organic molecule; adding the organic molecule onto the carrier; outputting a microwave energy by a coaxial cable; and emitting the microwave energy through a radiator to microwave the carrier and the organic molecule uniformly to coat the organic molecule onto the carrier carrying the silane, wherein the measuring kit is used to apply an enzyme-linked immunosorbent assay.

In accordance with another aspect of the present invention, a method for manufacturing a measuring kit is disclosed. The method for manufacturing a measuring kit includes steps of providing a carrier including a substrate having a surface; coating a silane onto the surface of the substrate; providing an organic molecule; adding the organic molecule onto the carrier; outputting a microwave energy by a coaxial cable; and emitting the microwave energy through a radiator uniformly to microwave the carrier and the organic molecule to coat the organic molecule onto the carrier carrying the silane.

In accordance with a further aspect of the present invention, a method for accelerating an attachment of an organic molecule to a carrier is disclosed. The method for accelerating an attachment of an organic molecule to a carrier includes steps of providing the carrier including a substrate having a surface; coating a silane onto the surface of the substrate; adding the organic molecule onto the carrier; and increasing a collision probability between the carrier carrying the silane and the organic molecule with a specific power to accelerate the attachment of the organic molecule to the carrier carrying the silane.

### BRIEF DESCRIPTION OF THE DRAWINGS

FIG. 1 shows comparison of coating transforming growth factor beta 1 (TGF-beta 1) overnight onto Corning's COR-9018 plate and coating TGF-beta 1 by microwaving in a microwave oven onto Corning's COR-9018 plate;

FIG. 2 shows comparison of coating TGF-beta 1 overnight onto Corning's COR-9018 plate and coating TGF-beta 1 by a microwave therapy apparatus onto Corning's COR-9018 plate;

FIGS. 3(a) and 3(b) show comparison of coating IL-15 by microwaving in a microwave oven onto a glass substrate carrier bearing gold nanoparticles and silane of the present invention and onto Corning's COR-9018 plate;

FIGS. 4(a) and 4(b) show comparison of coating TGF-beta 1 by microwaving in a microwave oven onto a glass substrate carrier bearing gold nanoparticles and silane of the present invention and onto Corning's COR-9018 plate;

FIG. 5 shows comparison of coating TGF-beta 1 overnight onto a glass substrate carrier bearing gold nanoparticles and silane of the present invention and coating TGF-beta 1 by a microwave therapy apparatus onto a glass substrate carrier bearing gold nanoparticles and silane of the present invention;

FIGS. 6(a) and 6(b) show comparison of coating TGF-beta 1 by a microwave therapy apparatus onto a glass substrate carrier bearing gold nanoparticles and silane of the present invention and coating TGF-beta 1 by a commercially available microwave oven onto a glass substrate carrier bearing gold nanoparticles and silane of the present invention;

FIG. 7 shows comparison of coating TGF-beta 1 by a microwave therapy apparatus onto a glass substrate carrier bearing gold nanoparticles and silane of the present invention and coating TGF-beta 1 by a microwave therapy apparatus onto Corning's COR-9018 plate;

FIGS. 8(a) and 8(b) show comparison of coating Interferon-gamma (IFN-gamma) by a microwave therapy apparatus onto a glass substrate carrier bearing gold nanoparticles and silane of the present invention and coating IFN-gamma by a microwave therapy apparatus onto Corning's COR-9018 plate;

FIGS. 9(a) and 9(b) show comparison of coating GM-CSF by a microwave therapy apparatus onto a glass substrate carrier bearing gold nanoparticles and silane of the present invention and coating GM-CSF by a microwave therapy apparatus onto Corning's COR-9018 plate;

FIGS. 10(a) and 10(b) show comparison of coating IL-12 by a microwave therapy apparatus onto a glass substrate carrier bearing gold nanoparticles and silane of the present invention and coating IL-12 by a microwave therapy apparatus onto Corning's COR-9018 plate;

FIG. 11 shows comparison of coating tumor necrosis factor-α (TNF-α) by a microwave therapy apparatus at different times onto a glass substrate carrier bearing gold nanoparticles and silane of the present invention;

FIG. 12 shows comparison of coating TGF-beta 1 by a microwave therapy apparatus onto a glass substrate carrier bearing gold nanoparticles and silane of the present invention and onto a glass substrate carrier bearing silane of the present invention; and

FIG. 13 shows comparison of coating TGF-beta 1 by a microwave therapy apparatus onto a glass substrate carrier bearing gold nanoparticles of the present invention and onto a glass substrate carrier bearing silane of the present invention.

### DETAILED DESCRIPTION OF THE PREFERRED EMBODIMENTS

The present invention will now be described more specifically with reference to the following embodiments. It is to be noted that the following descriptions of preferred embodiments of this invention are presented herein for the purposes of illustration and description only; they are not intended to be exhaustive or to be limited to the precise form disclosed.

The first embodiment of the present invention provides a method for manufacturing a measuring kit. The method for manufacturing a measuring kit includes steps of providing a carrier including a substrate having a surface; coating a silane onto the surface of the substrate; providing an organic molecule; adding the organic molecule onto the carrier; outputting a microwave energy by a coaxial cable; and emitting the microwave energy through a radiator to microwave the carrier and the organic molecule uniformly to coat the organic molecule onto the carrier carrying the silane, wherein the measuring kit is used to apply an enzyme-linked immunosorbent assay.

The carrier in this embodiment may be but is not limited to a chip, a strip or a plate. The chip may be a microarray or a lab-on-a-chip. The microarray may be but is not limited to a protein chip, a gene chip and a microchannel chip. The silane may be an alkylsilane, aminosilane or other silane. The aminosilane may be, for example, a 3-aminopropryltrimethoxysilane (APTMS) or 3-aminopropyltriethoxysilane (APTS). This embodiment may use any means or apparatus capable of outputting a microwave energy by a coaxial cable and emitting the microwave energy through a radiator uniformly. For example, a microwave therapy apparatus may be used, but is not limited thereto. In this embodiment, the carrier carrying the silane and the organic molecule may be microwaved by a power of less than 200 W. The microwaving step is more preferably performed under 60 W for 30-40 minutes, and the best is 60 W for 30 minutes.

The carrier in this embodiment may bears a plurality of spaced metal nanoparticles. The metal nanoparticles may be gold nanoparticles. The substrate of the carrier in this embodiment may be made of one of a glass and a plastic, and the glass may be an optical glass.

The enzyme-linked immunosorbent assay in this embodiment may be one selected from a group consisting of a sandwich enzyme-linked immunosorbent assay, an indirect enzyme-linked immunosorbent assay and a competitive enzyme-linked immunosorbent assay. The organic molecule may be one of an antibody and an antigen.

The second embodiment of the present invention provides a method for manufacturing a measuring kit. The method for manufacturing a measuring kit includes steps of providing a carrier including a substrate having a surface; coating a silane onto the surface of the substrate; providing an organic molecule; adding the organic molecule onto the carrier; outputting a microwave energy by a coaxial cable; and emitting the microwave energy through a radiator uniformly to microwave the carrier and the organic molecule to coat the organic molecule onto the carrier carrying the silane.

The carrier in this embodiment may be but is not limited to a chip, a strip or a plate. The chip may be a microarray or a lab-on-a-chip. The microarray may be but is not limited to a protein chip, a gene chip and a microchannel chip. The silane may be an alkylsilane, aminosilane or other silane. The aminosilane may be, for example, a 3-aminopropryltrimethoxysilane (APTMS) or 3-aminopropyltriethoxysilane (APTS). This embodiment may use any means or apparatus capable of outputting a microwave energy by a coaxial cable and emitting the microwave energy through a radiator uniformly. For example, a microwave therapy apparatus may be used, but is not limited thereto. In this embodiment, the carrier carrying the silane and the organic molecule may be microwaved by a power of less than 200 W. The microwaving step is more preferably performed under 60 W for 30-40 minutes, and the best is 60 W for 30 minutes.

The carrier in this embodiment may bear a plurality of spaced metal nanoparticles. The metal nanoparticles may be gold nanoparticles. The substrate of the carrier in this embodiment may be made of one of a glass and a plastic, and the glass may be an optical glass.

The measuring kit in this embodiment is used to apply to a measuring method. The measuring method may be one selected from a group consisting of a sandwich enzyme-linked immunosorbent assay, an indirect enzyme-linked immunosorbent assay and a competitive enzyme-linked immunosorbent assay. The organic molecule may be one of an antibody and an antigen.

The third embodiment of the present invention provides a method for accelerating an attachment of an organic molecule to a carrier is disclosed. The method for accelerating an attachment of an organic molecule to a carrier includes steps of providing the carrier including a substrate having a surface; coating a silane onto the surface of the substrate; adding the organic molecule onto the carrier; and increasing a collision probability between the carrier carrying the silane and the organic molecule with a specific power to accelerate the attachment of the organic molecule to the carrier carrying the silane.

The method for accelerating an attachment of an organic molecule to a carrier in this embodiment, wherein the specific power is provided when a microwave energy is outputted by a coaxial cable and is uniformly emitted through a radiator to microwave the carrier carrying the silane and the organic molecule. The specific power has a fixed value. The fixed value means that the specific power is kept constant and does not change with changes in voltage or current.

The carrier in this embodiment may be but is not limited to a chip, a strip or a plate. The chip may be a microarray or a lab-on-a-chip. The microarray may be but is not limited to a protein chip, a gene chip and a microchannel chip. The silane may be an alkylsilane, aminosilane or other silane. The aminosilane may be, for example, a 3-aminopropryltrimethoxysilane (APTMS) or 3-aminopropyltriethoxysilane (APTS). This embodiment may use any means or apparatus capable of outputting a microwave energy by a coaxial cable and emitting the microwave energy through a radiator uniformly. For example, a microwave therapy apparatus may be used, but is not limited thereto. In this embodiment, the carrier carrying the silane and the organic molecule may be microwaved by a power of less than 200 W. The microwaving step is more preferably performed under 60 W for 30-40 minutes, and the best is 60 W for 30 minutes.

The carrier in this embodiment may bear a plurality of spaced metal nanoparticles. The metal nanoparticles may be gold nanoparticles. The substrate of the carrier in this embodiment may be made of one of a glass and a plastic, and the glass may be an optical glass.

The method for accelerating an attachment of an organic molecule to a carrier in this embodiment is used to apply to a measuring method. The measuring method may be one selected from a group consisting of a sandwich enzyme-linked immunosorbent assay, an indirect enzyme-linked immunosorbent assay and a competitive enzyme-linked immunosorbent assay. The organic molecule may be one of an antibody and an antigen.

In a traditional enzyme-linked immunosorbent assay, coating proteins (antibodies or antigens) to plastic plate is mainly by standing. It uses electrostatic adsorption to coat the proteins on the surface of the plastic plate. Coating usually takes 12-18 hours and the time is too long. Therefore, the present invention provides a coating technique that outputs a microwave energy by a coaxial cable and emits the microwave energy through a radiator uniformly to solve the shortcomings of the disadvantage of the long coating time of the traditional enzyme-linked immunosorbent assay.

The principle of the present invention is to output a microwave energy by a way of a low-power coaxial cable and emit the microwave energy (<200 W) through a radiator uniformly to treat a coating buffer (containing proteins such as antibodies or antigens) in the carrier. This causes the water molecules in the coating buffer to oscillate at a frequency of 245 million times per second, resulting in an increase in the frequency of collisions between the protein molecules in the coating buffer, thereby increasing the collision probability between the protein molecules and the surface of the carrier, enabling the protein molecules to adsorb on the surface of the carrier in a short time, and the detection sensitivity is not reduced accordingly. In addition, the overall temperature of the carrier and the coating buffer in the invention after the microwave treatment only increases by about 3-4 degrees, which can prevent the protein molecules from denaturing due to the high temperature.

On the other hand, general commercially available microwave ovens use a magnetron to generate microwaves, and then use a waveguide to output microwaves to drive into a resonant cavity and perform microwave heating by a way of cavity resonance. Such a microwave energy output is likely to fluctuate with an external power supply. Therefore, the intensity of the microwave energy in the resonant cavity is directly affected, so that stable microwave power cannot be obtained. In addition, when the microwave oven is operating, the microwaves will form standing waves in the cavity, so the turntable needs to be continuously rotated so that the energy can be evenly distributed. However, it is still difficult to make the energy evenly distributed only by rotating the turntable. In the case of uneven microwave energy intensity received by the carrier, microwave coating using a microwave oven can only be performed for a short period of time, such as 20 seconds used in the present application, to avoid protein denaturation such as antibodies loss of activity. However, if the microwave time is too short, the coating effect is poor and the OD value is lower in the areas that receive less microwave energy intensity. Therefore, the use of microwave oven microwaves to coat protein molecules will lead to unstable experimental data and the OD value is relatively non-reproducible.

Therefore, according to the microwave coating used in the present invention, the microwave source feeds microwaves to a rectangular output plate (radiation probe) in a manner that a coaxial cable outputs microwave energy, and the size of the rectangular output plate (radiation probe) coincides with the 96-well plate, the two fit perfectly when microwaving, so every corner of the plate can be microwaved evenly. In addition, a stable microwave power output has the advantage of outputting a microwave energy by a coaxial cable and emitting the microwave energy through a radiator uniformly, and the negative feedback technology of a PID (proportional-integral-derivative control) can be designed to effectively ensure that the output microwave power does not fluctuate with external power supply fluctuations. Therefore, coating by using a way of outputting a microwave energy by a coaxial cable and emitting the microwave energy through a radiator uniformly can obtain stable experimental data.

Therefore, the method for manufacturing a measuring kit and the method for accelerating an attachment of an organic molecule to a carrier of the present invention using a way of outputting a microwave energy by a coaxial cable and emitting the microwave energy through a radiator uniformly greatly reduce the time required for the enzyme-linked immunosorbent assay, do not affect the detection sensitivity, and can obtain stable experimental data.

The ELISA in the experiments of the present invention is carried out by the following steps:

Solutions:

Wash buffer-0.05% Tween 20 in Phosphate buffered saline (PBS)
5× Diluent-1% Bovine serum albumin (BSA) in PBS
Substrate solution-1×TMB
Stop Solution-2N H₂SO₄
10× Coating buffer

Materials:

Capture antibody: dilute 10× Coating buffer with ddH₂O to 1× Coating buffer. Then dilute Capture antibody with 1× Coating buffer to 1× Capture antibody.
Standard: dilute the target protein standard with 1× Diluent to the highest concentration, and then carry out serial dilution.
Detection antibody: Detection antibody is diluted 250-fold with 1× Diluent to 1× Detection antibody.
Avidin-HRP: Avidin-HRP is diluted 250-fold with 1× Diluent to 1× Avidin-HRP.

Process:

Step 1:

a. dilute capture antibody with 1× coating buffer, add 100 μl/well of the diluted capture antibody to a glass/plastic strip or plate with/without gold nanoparticles and silane on the surface, seal the plate and stand the plate at 4° C. overnight (O/N).

b. coating by a commercially available microwave oven: capture antibody is diluted 250-fold with 1× coating buffer for the coating step. Add 100 μl/well capture antibody to a glass/plastic strip or plate with/without gold nanoparticles and silane on the surface, microwave at 80 W for 20 seconds, and stand for 10 minutes at room temperature (replacing the traditional plate that needs to stand at 4° C. overnight).

c. coating by outputting a microwave energy by a coaxial cable and emitting the microwave energy through a radiator uniformly: capture antibody is diluted 250-fold with 1× coating buffer for the coating step. Add 100 μl/well capture antibody to a glass/plastic strip or plate with/without gold nanoparticles and silane on the surface, and microwave at 60 W for 30 minutes (replacing the traditional plate that needs to stand at 4° C. overnight).

Step 2: Wash the plate four times with Wash buffer of more than 250 μl/well. Soak for 1 minute each time after washing, and flip down on the paper towel to remove residual liquid.

Step 3: Add 100 μl/well of 1× Diluent and stand at room temperature for 1 hour.

Step 4: Wash the plate four times with Wash buffer of more than 250 μl/well. Soak for 1 minute each time after washing, and flip down on the paper towel to remove residual liquid.

Step 5: Serially dilute the standard with 1× Diluent and add 100 μl/well to the plate and stand at room temperature for 2 hours.

Step 6: Wash the plate four times with Wash buffer of more than 250 μl/well. Soak for 1 minute each time after washing, and flip down on the paper towel to remove residual liquid.

Step 7: Dilute the Detection antibody with 1× Diluent, add 100 μl/well and stand at room temperature for 1 hour.

Step 8: Wash the plate six times with Wash buffer of more than 250 μl/well. Soak for 1 minute each time after washing, and flip down on the paper towel to remove residual liquid.

Step 9: Dilute Avidin-HRP with 1× Diluent, add 100 μl/well, and stand in the dark for 30 minutes at room temperature.

Step 10: Wash the plate six times with Wash buffer of more than 250 μl/well. Soak for 1 minute each time after washing, and flip down on the paper towel to remove residual liquid.

Step 11: Add 100 μl/well TMB substrate and protect from light for 15 minutes for coloration.

Step 12: Add 50 μl/well H₂SO₄ to stop the reaction.

Step 13: Use an ELASA analyzer to measure the results at 450 nm and correct the results at 630 nm.

Experiments

1. Comparison of coating transforming growth factor beta 1 (TGF-beta 1) overnight onto Corning's COR-9018 plate and coating TGF-beta 1 by microwave in a microwave oven onto Corning's COR-9018 plate:

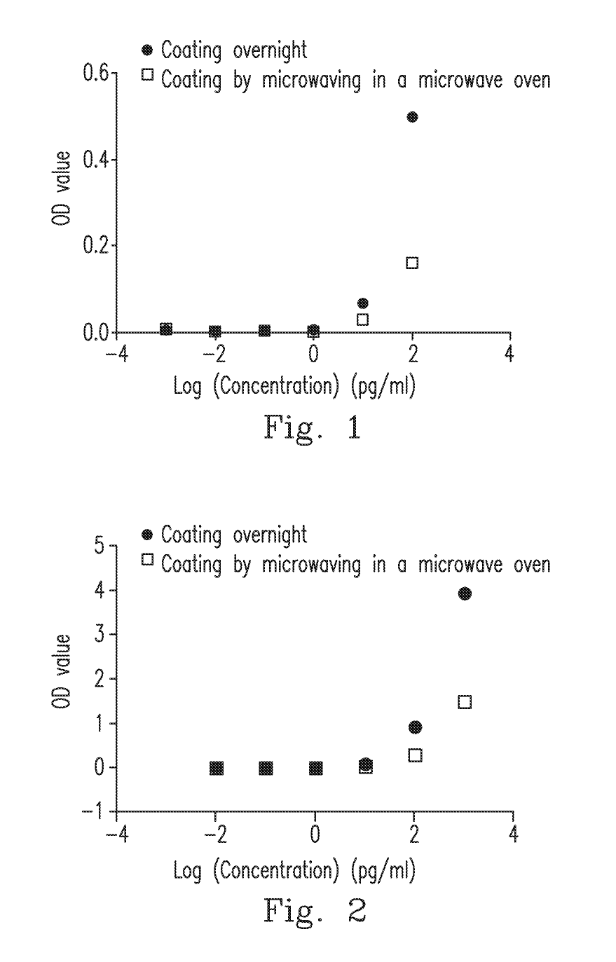

Please refer to FIG. 1 and Table 1. Use commercially available microwave oven to coat TGF-beta 1 onto Corning's COR-9018 plate (which is a commercially available plastic plate) by a power of 10% (about 80 W) for 20 seconds. In the result, not only sensitivity is reduced but also OD values are decreased. Coating by microwaving in a microwave oven fails to improve the effectiveness of COR-9018 plate for coating TGF-beta 1.

TABLE 1

OD value

| Concentration | Coating | Coating by microwaving |
|---|---|---|
| (pg/ml) | overnight | in a microwave oven |
| 100 | 0.49930 | 0.162 |
| 10 | 0.06727 | 0.030 |
| 1 | 0.00688 | 0.002 |
| 0.1 | 0.00279 | 0.003 |
| 0.01 | 0.00163 | 0.003 |
| 0.001 | 0.00350 | 0.007 |

2. Comparison of coating TGF-beta 1 overnight onto Corning's COR-9018 plate and coating TGF-beta 1 by a microwave therapy apparatus onto Corning's COR-9018 plate:

Please refer to FIG. 2 and Table 2. Use a microwave therapy apparatus to coat TGF-beta 1 onto Corning's COR-9018 plate (which is a commercially available plastic plate). In the result, not only sensitivity is reduced but also OD values are decreased. Coating by a microwave therapy apparatus fails to improve the effectiveness of COR-9018 plate for coating TGF-beta 1.

TABLE 2

OD value

| Concentration | Coating | Coating by a microwave |
|---|---|---|
| (pg/ml) | overnight | therapy apparatus |
| 1000 | Exceed the | 1.5040 |
| | measurable | |
| | limit | |
| 100 | 0.9837 | 0.3166 |
| 10 | 0.1418 | 0.0442 |
| 1 | 0.0609 | 0.0185 |
| 0.1 | 0.0541 | 0.0158 |
| 0.01 | 0.0649 | 0.0170 |
| Blank | 0.0613 | 0.0246 |
| control | | |
| group | | |

3. Comparison of coating IL-15 by microwaving in a microwave oven onto a glass substrate carrier bearing gold nanoparticles and silane of the present invention and onto Corning's COR-9018 plate:

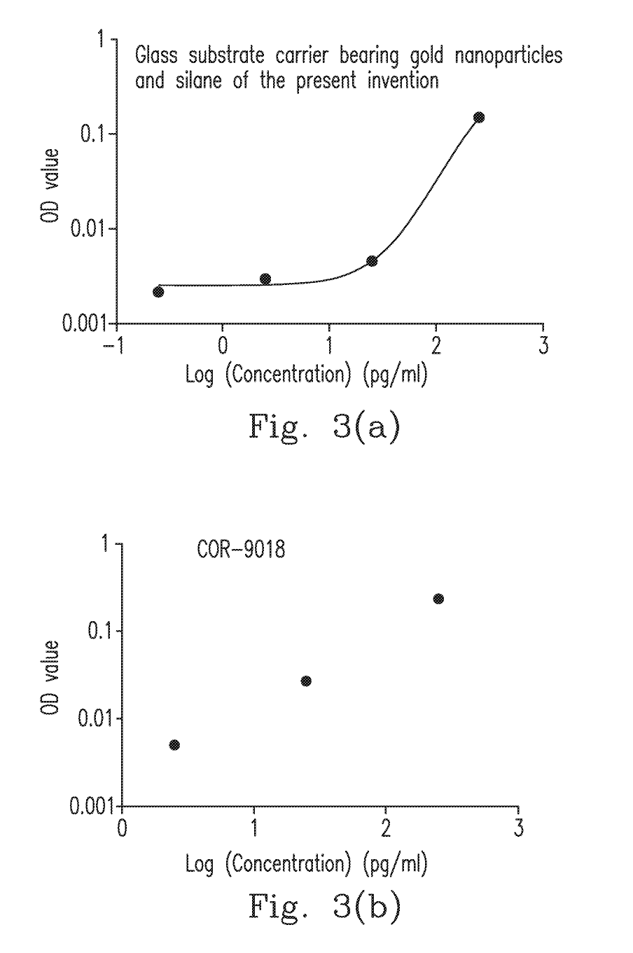

Please refer to FIG. 3(a), FIG. 3(b) and Table 3. Use commercially available microwave oven to coat IL-15 onto the carrier of the present invention and Corning's COR-9018 plate by a power of 10% (about 80 W) for 20 seconds. After microwave coating, the sensitivity of IL-15 of the carrier of the present invention is 0.25 pg/ml, and the sensitivity of IL-15 of Corning's COR-9018 plate is 2.5 pg/ml. The sensitivity of the carrier of the present invention exceeds the traditional plate by more than 10 times.

TABLE 3

OD value

| | The glass substrate carrier | |
|---|---|---|
| Concentration | bearing gold nanoparticles and | |
| (pg/ml) | silane of the present invention | COR-9018 |
| 2500 | Exceed the | Exceed the |
| | measurable | measurable |
| | limit | limit |
| 250 | 0.2821 | 0.2359 |
| 25 | 0.0277 | 0.0276 |
| 2.5 | 0.0207 | 0.0051 |
| 0.25 | 0.0168 | 0.0073 |

4. Comparison of coating TGF-beta 1 by microwaving in a microwave oven onto a glass substrate carrier bearing gold nanoparticles and silane of the present invention and onto Corning's COR-9018 plate:

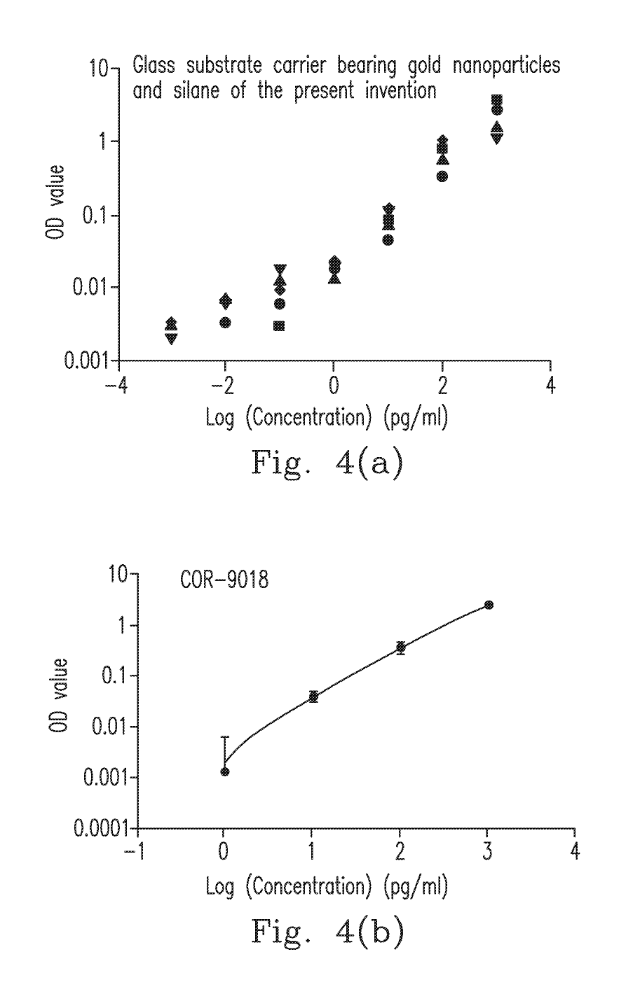

Please refer to FIG. 4(a), FIG. 4(b) and Table 4. Use commercially available microwave oven to coat TGF-beta 1 onto the carrier of the present invention and Corning's COR-9018 plate by a power of 10% (about 80 W) for 20 seconds. After microwave coating using commercially available microwave, the best sensitivity of TGF-beta 1 of the carrier of the present invention can reach 0.001 pg/ml, and the sensitivity of TGF-beta 1 of Corning's COR-9018 plate is 10-1 pg/ml. After microwave coating by a commercially available microwave, the sensitivity of the carrier of the present invention exceeds the traditional plate by more than 10 times. However, due to the unstable output power of the microwave oven, the consistency of the OD value is not good.

TABLE 4

| | OD value | | | | | | |
|---|---|---|---|---|---|---|---|
| | The glass substrate carrier | | | | | | |
| Concentration | bearing gold nanoparticles and | | | | | | |
| (pg/ml) | silane of the present invention | | | | | COR-9018 | |
| 1000 | 2.7204 | 3.7078 | 1.5913 | 1.08807 | Exceed the | 2.1886 | 2.5786 |
| | | | | | measurable | | |
| | | | | | limit | | |
| 100 | 0.3331 | 0.8055 | 0.5864 | 0.78117 | 1.05845 | 0.2908 | 0.4125 |
| 10 | 0.0466 | 0.0854 | 0.0744 | 0.11387 | 0.12495 | 0.0331 | 0.0444 |
| 1 | 0.0179 | 0.0193 | 0.0138 | 0.02018 | 0.02383 | −0.0022 | 0.0048 |
| 0.1 | 0.0062 | 0.0031 | 0.0131 | 0.01658 | 0.00959 | −0.0022 | 0.0013 |
| 0.01 | 0.0034 | 0.0003 | 0.0072 | 0.00638 | 0.006 | −0.0034 | 0.0002 |
| 0.001 | −0.0014 | 3.7E−10 | 0.0032 | 0.00200 | 0.00341 | −0.0037 | 0.0002 |

5. Comparison of coating TGF-beta 1 overnight onto a glass substrate carrier bearing gold nanoparticles and silane of the present invention and coating TGF-beta 1 by microwaving with a microwave therapy apparatus onto a glass substrate carrier bearing gold nanoparticles and silane of the present invention:

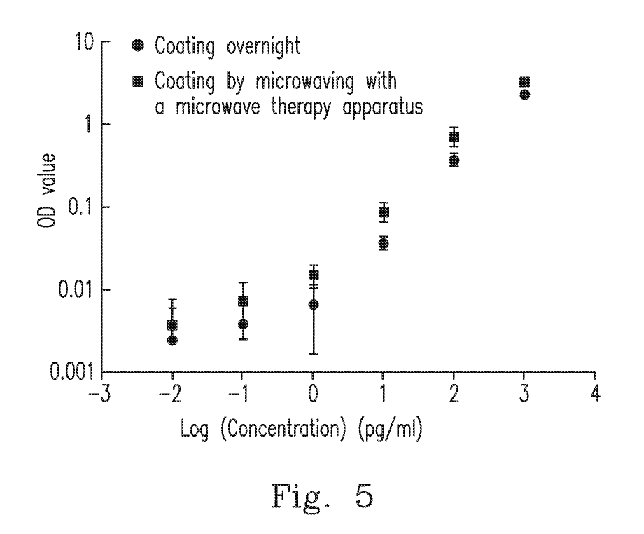

Please refer to FIG. 5 and Table 5. Coating TGF-beta 1 overnight and coating TGF-beta 1 by microwaving with a microwave therapy apparatus onto carriers bearing gold nanoparticles and silane of the present invention obtain similar OD values, and both reach sensitivity of 1 pg/ml.

TABLE 5

| Concen- | | | | | |
|---|---|---|---|---|---|
| tration | OD value | | | | |
| (pg/ml) | Coating overnight | | Coating by a microwave therapy apparatus | | |
| 1000 | 2.3771 | 2.5472 | Exceed the | 3.470 | Exceed the |
| | | | measurable | | measurable |
| | | | limit | | limit |
| 100 | 0.4691 | 0.3902 | 0.8538 | 0.8984 | 0.6088 |
| 10 | 0.0839 | 0.0733 | 0.1400 | 0.1735 | 0.1343 |
| 1 | 0.0527 | 0.0428 | 0.07396 | 0.07416 | 0.0807 |
| 0.1 | 0.0485 | 0.0414 | 0.06580 | 0.06643 | 0.0735 |
| 0.01 | 0.0475 | 0.0397 | 0.06413 | 0.06232 | 0.068 |
| Blank | 0.0426 | 0.0368 | 0.06051 | 0.06253 | 0.0604 |
| control | | | | | |
| group | | | | | |

6. Comparison of coating TGF-beta 1 by a microwave therapy apparatus onto a glass substrate carrier bearing gold nanoparticles and silane of the present invention and coating TGF-beta 1 by a commercially available microwave oven onto a glass substrate carrier bearing gold nanoparticles and silane of the present invention:

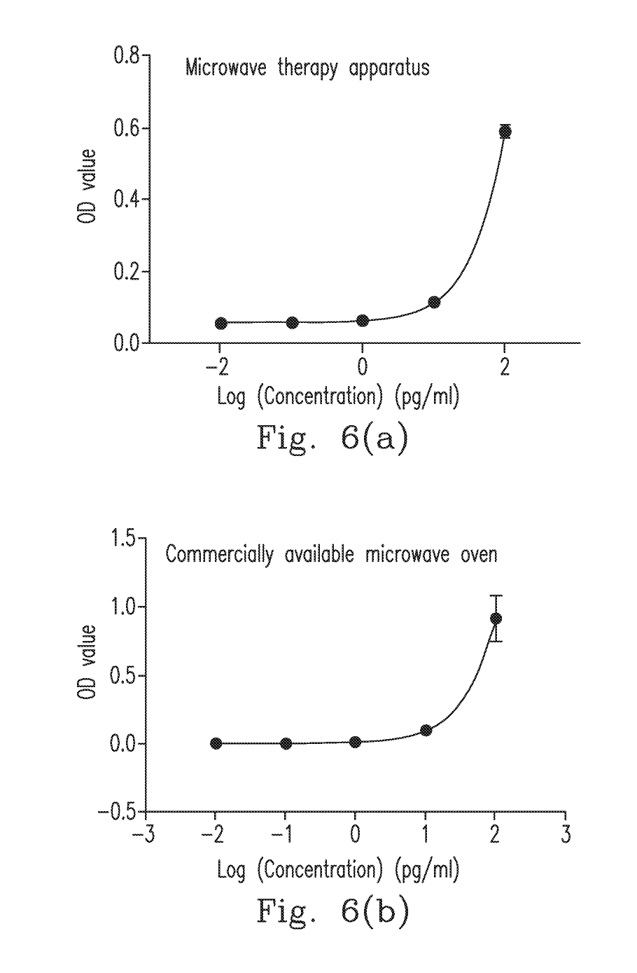

Please refer to FIGS. 6(a), 6(b) and Table 6. Carry out microwave coating experiments using a microwave therapy apparatus and a commercially available microwave oven. Microwave condition of the commercially available microwave oven is 10% of power for 20 seconds. Microwave condition of the microwave therapy apparatus is 60 W for 30 minutes. As a result, regarding microwave coating data of the commercially available microwave oven, because microwave ovens use a magnetron to generate microwaves, and then use a waveguide to input microwaves into a resonant cavity through, such a microwave energy output way causes microwave energy to be unstable and makes the OD value less reproducible, and the experimental results are not stable. Using a microwave therapy apparatus, i.e. a way of outputting a microwave energy by a coaxial cable and emitting the microwave energy through a radiator uniformly, the output power is stable, and uniform microwaves can be provided to the carrier without being affected by the position of the sample on the carrier, and the obtained OD value is stable.

TABLE 6

| Concentration | OD value | | | | | |
|---|---|---|---|---|---|---|
| (pg/ml) | Microwave therapy apparatus | | | Commercially available microwave oven | | |
| 100 | 0.597 | 0.6002 | 0.5701 | 0.9334 | 1.138 | 0.7701 |
| 10 | 0.1144 | 0.1156 | 0.1183 | 0.1226 | 0.1577 | 0.1113 |
| 1 | 0.07187 | 0.06281 | 0.05968 | 0.03501 | 0.07398 | 0.03680 |
| 0.1 | 0.06893 | 0.05742 | 0.05425 | 0.02383 | 0.05378 | 0.03045 |
| 0.01 | 0.06637 | 0.05560 | 0.05391 | 0.02286 | 0.05999 | 0.02919 |
| Blank | 0.06581 | 0.06012 | 0.05381 | 0.02942 | 0.11584 | 0.03363 |
| control | | | | | | |
| group | | | | | | |

7. Comparison of coating TGF-beta 1 by a microwave therapy apparatus onto a glass substrate carrier bearing gold nanoparticles and silane of the present invention and coating TGF-beta 1 by a microwave therapy apparatus onto Corning's COR-9018 plate:

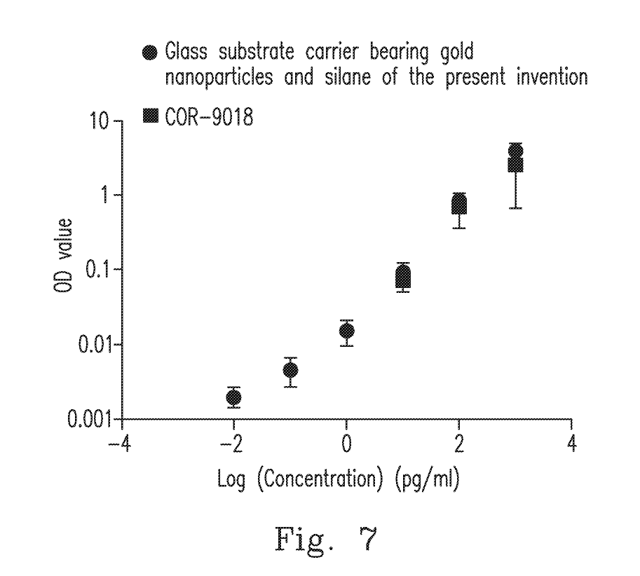

Please refer to FIG. 7 and Table 7. Microwave a carrier of the present invention and Corning's COR-9018 plate at 60 W for 30 minutes by a microwave therapy apparatus to coat TGF-beta 1. After coating by a microwave therapy apparatus, the best sensitivity of TGF-beta 1 of the carrier of the present invention can reach 0.1-0.01 pg/ml, and the sensitivity of TGF-beta 1 of Corning's COR-9018 plate is 10-1 pg/ml. After microwave coating by a microwave therapy apparatus, the sensitivity of the carrier of the present invention exceeds the traditional plate by more than 10 times, and the consistency of the OD value is good.

TABLE 7

| | OD value | | | |
|---|---|---|---|---|
| | The glass substrate carrier | | | |
| Concentration | bearing gold nanoparticles and | | | |
| (pg/ml) | silane of the present invention | | COR-9018 | |
| 1000 | Exceed the | 3.470 | Exceed the | 1.271 |
| | measurable | | measurable | |
| | limit | | limit | |
| 100 | 0.8538 | 0.8984 | 0.9800 | 0.4936 |
| 10 | 0.1400 | 0.1735 | 0.1298 | 0.08029 |
| 1 | 0.07396 | 0.07416 | 0.04148 | 0.02764 |
| 0.1 | 0.06580 | 0.06643 | 0.03645 | 0.01943 |
| 0.01 | 0.06413 | 0.06232 | 0.04387 | 0.01921 |
| Blank | 0.06051 | 0.06253 | 0.05533 | 0.02324 |
| control | | | | |
| group | | | | |

8. Comparison of coating Interferon-gamma (IFN-gamma) by a microwave therapy apparatus onto a glass substrate carrier bearing gold nanoparticles and silane of the present invention and coating IFN-gamma by a microwave therapy apparatus onto Corning's COR-9018 plate:

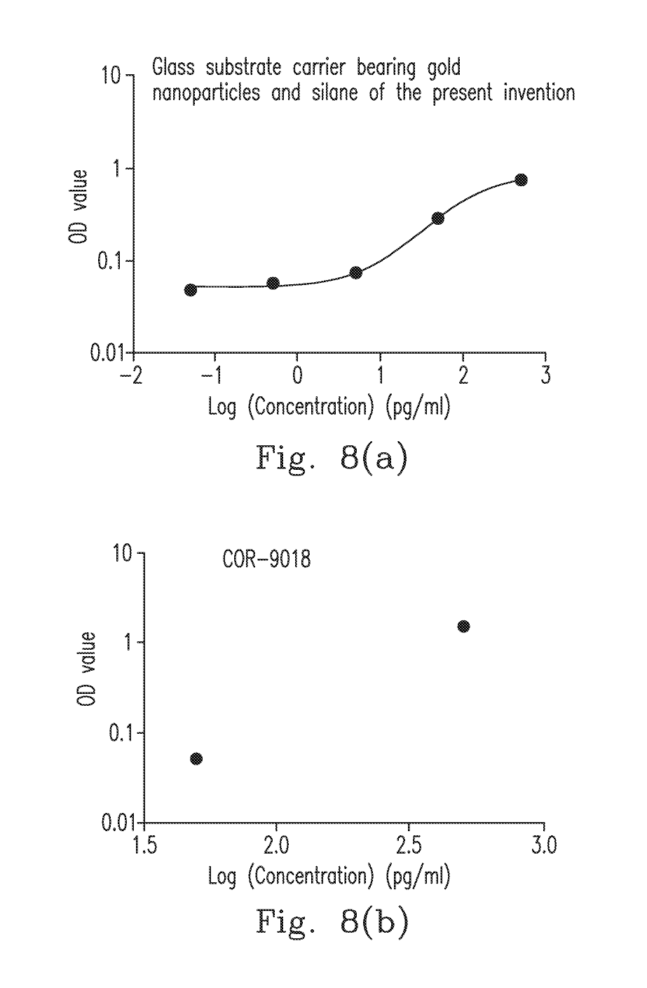

Please refer to FIGS. 8(a), 8(b) and Table 8. After coating by a microwave therapy apparatus, the best sensitivity of IFN-gamma of the carrier of the present invention is 0.05 pg/ml, and the sensitivity of IFN-gamma of Corning's COR-9018 plate is 50-5 pg/ml. The sensitivity of the carrier of the present invention exceeds Corning's COR-9018 plate by more than 100 times.

TABLE 8

OD value

| | The glass substrate carrier | |
|---|---|---|
| Concentration | bearing gold nanoparticles and | |
| (pg/ml) | silane of the present invention | COR-9018 |
| 500 | 0.7406 | 1.501 |
| 50 | 0.2882 | 0.052 |
| 5 | 0.0739 | −0.0409 |
| 0.5 | 0.0574 | −0.0586 |
| 0.05 | 0.0485 | 0.0587 |

9. Comparison of coating GM-CSF by a microwave therapy apparatus onto a glass substrate carrier bearing gold nanoparticles and silane of the present invention and coating GM-CSF by a microwave therapy apparatus onto Corning's COR-9018 plate:

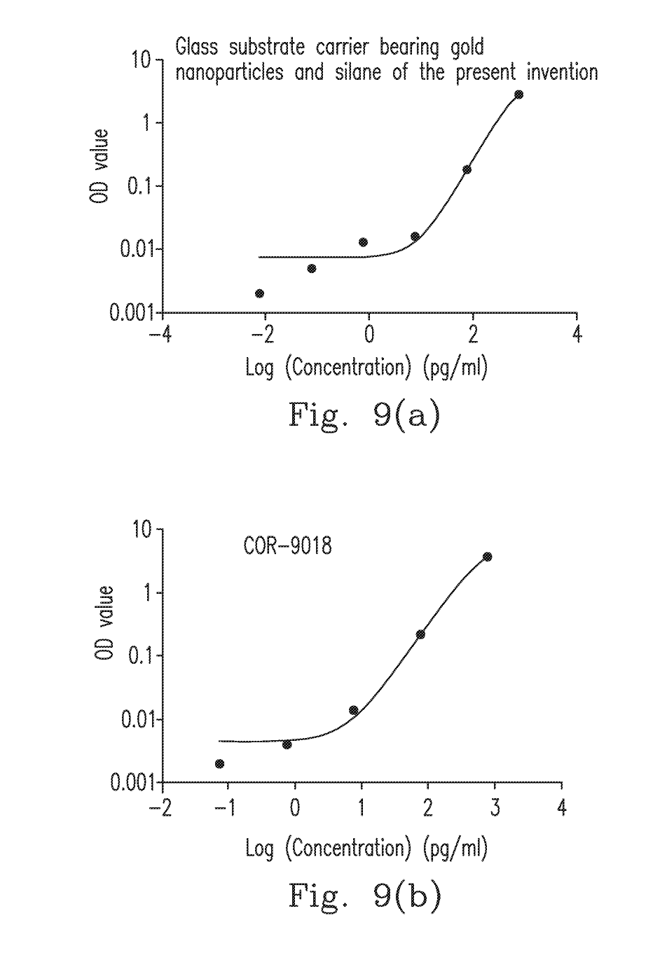

Please refer to FIGS. 9(a), 9(b) and Table 9. After coating by a microwave therapy apparatus, the best sensitivity of GM-CSF of the carrier of the present invention can reach 0.0075 pg/ml, and the sensitivity of GM-CSF of Corning's COR-9018 plate is 0.075 pg/ml. The sensitivity of the carrier of the present invention exceeds Corning's COR-9018 plate by more than 10 times.

TABLE 9

OD value

| | The glass substrate carrier | |
|---|---|---|
| Concentration | bearing gold nanoparticles and | |
| (pg/ml) | silane of the present invention | COR-9018 |
| 750 | 2.782 | 3.634 |
| 75 | 0.182 | 0.217 |
| 7.5 | 0.016 | 0.014 |
| 0.75 | 0.013 | 0.004 |
| 0.075 | 0.005 | 0.002 |
| 0.0075 | 0.002 | −0.001 |
| 0.00075 | −0.003 | −0.002 |

10. Comparison of coating IL-12 by a microwave therapy apparatus onto a glass substrate carrier bearing gold nanoparticles and silane of the present invention and coating IL-12 by a microwave therapy apparatus onto Corning's COR-9018 plate:

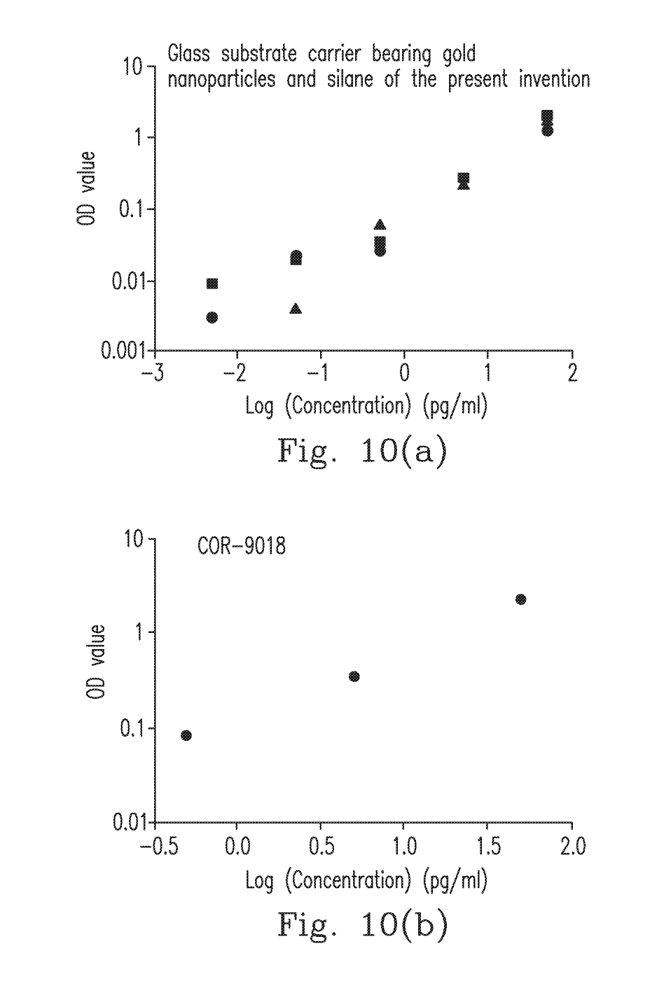

Please refer to FIGS. 10(a), 10(b) and Table 10. After coating by a microwave therapy apparatus, the best sensitivity of IL-12 of the carrier of the present invention can reach 0.005 pg/ml, and the sensitivity of IL-12 of Corning's COR-9018 plate is 0.5 pg/ml. The sensitivity of the carrier of the present invention exceeds Corning's COR-9018 plate by more than 100 times.

TABLE 10

| | OD value | | | |
|---|---|---|---|---|
| Concen- | The glass substrate carrier | | | |
| tration | bearing gold nanoparticles and | | | |
| (pg/ml) | silane of the present invention | | COR-9018 | |
| 500 | Exceed the | Exceed the | Exceed the | Exceed the |
| | measurable | measurable | measurable | measurable |
| | limit | limit | limit | limit |
| 50 | 1.246 | 2.057 | 1.771 | 2.273681 |
| 5 | 0.264 | 0.278 | 0.222 | 0.347281 |
| 0.5 | 0.026 | 0.035 | 0.061 | 0.082051 |
| 0.05 | 0.022 | 0.020 | 0.004 | 0.103181 |
| 0.005 | 0.003 | 0.009 | 0.0003 | 0.044191 |

11. Comparison of coating tumor necrosis factor-α (TNF-α) by a microwave therapy apparatus at different times onto a glass substrate carrier bearing gold nanoparticles and silane of the present invention:

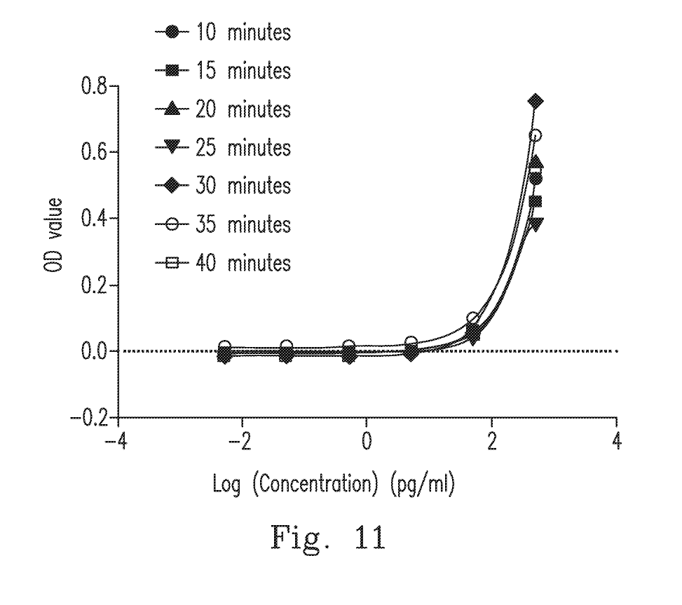

Please refer to FIG. 11 and Table 11. Microwave by a microwave therapy apparatus at different times, i.e. for 10, 15, 20, 25, 30, 35 and 40 minutes, respectively. The results show that the microwaving for 30 minutes can obtain the maximum linearity, achieve a better detection curve, space out the concentration detection range, and get the best results.

TABLE 11

| TNF-alpha | 10 | 15 | 20 | 25 | 30 | 35 | 40 |
|---|---|---|---|---|---|---|---|
| (pg/ml) | minutes | minutes | minutes | minutes | minutes | minutes | minutes |
| 500 | 0.5843 | 0.5143 | 0.6321 | 0.4480 | 0.8289 | 0.7048 | 0.6248 |
| 50 | 0.1216 | 0.1257 | 0.1347 | 0.1088 | 0.1495 | 0.1549 | 0.1268 |
| 5 | 0.07072 | 0.06509 | 0.06718 | 0.06485 | 0.06767 | 0.07962 | 0.07701 |
| 0.5 | 0.06480 | 0.05875 | 0.06470 | 0.06315 | 0.06108 | 0.06748 | 0.07062 |
| 0.05 | 0.06334 | 0.05901 | 0.06051 | 0.06128 | 0.06126 | 0.06946 | 0.06817 |
| 0.005 | 0.06409 | 0.06038 | 0.06289 | 0.06213 | 0.06172 | 0.06724 | 0.06811 |
| Blank | 0.06198 | 0.06034 | 0.06202 | 0.06202 | 0.07127 | 0.05117 | 0.07040 |
| control | | | | | | | |
| group | | | | | | | |
| Blank | 0.06298 | 0.06228 | 0.04538 | 0.06346 | 0.06981 | 0.05117 | 0.07178 |
| control | | | | | | | |
| group | | | | | | | |

12. Comparison of coating TGF-beta 1 by a microwave therapy apparatus onto a glass substrate carrier bearing gold nanoparticles and silane of the present invention and onto a glass substrate carrier bearing silane of the present invention:

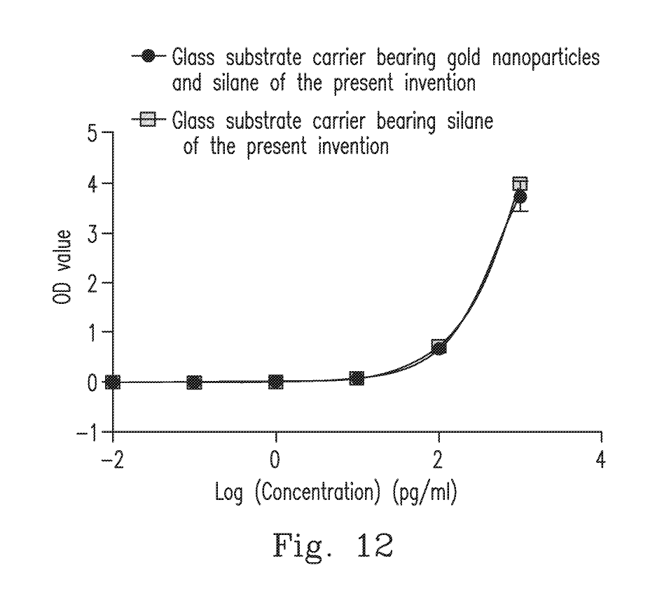

Please refer to FIG. 12 and Table 12. Compare a glass substrate carrier bearing gold nanoparticles and silane with a glass substrate carrier bearing silane after coating ten-fold diluted TGF-beta 1 standard by a microwave therapy apparatus. The overall OD values of the glass substrate carrier bearing gold nanoparticles and silane are higher, but after subtracting the background value, the glass substrate carrier bearing gold nanoparticles did not differ significantly from the glass substrate carrier bearing silane. After microwave coating by a microwave therapy apparatus, both of them can achieve good sensitivity.

TABLE 12

| Concen- | The glass substrate carrier | | The glass substrate | |
|---|---|---|---|---|
| tration | bearing gold nanoparticles and | | carrier bearing silane | |
| (pg/ml) | silane of the present invention | | of the present invention | |
| 1000 | Exceed the | 3.575 | Exceed the | Exceed the |
| | measurable | | measurable | measurable |
| | limit | | limit | limit |
| 100 | 0.7612 | 0.7255 | 0.7280 | 0.7535 |
| 10 | 0.1483 | 0.1481 | 0.09843 | 0.09924 |
| 1 | 0.08253 | 0.07239 | 0.02227 | 0.02465 |
| 0.1 | 0.07399 | 0.06639 | 0.01655 | 0.01717 |
| 0.01 | 0.07599 | 0.06307 | 0.01674 | 0.01646 |
| Blank | 0.07337 | 0.06284 | 0.01505 | 0.01659 |
| control | | | | |
| group | | | | |
| Blank | 0.07700 | 0.06322 | 0.01710 | 0.01672 |
| control | | | | |
| group | | | | |

13. Comparison of coating TGF-beta 1 by a microwave therapy apparatus onto a glass substrate carrier bearing gold nanoparticles of the present invention and onto a glass substrate carrier bearing silane of the present invention:

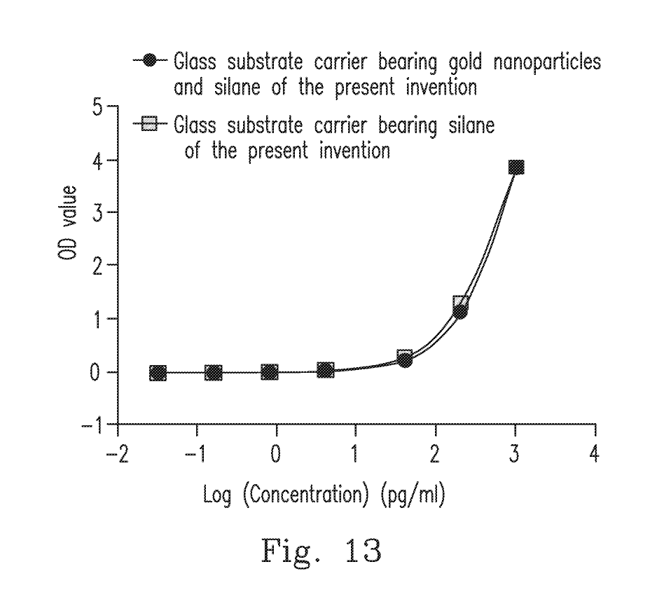

Please refer to FIG. 13 and Table 13. Compare a glass substrate carrier bearing gold nanoparticles and silane with a glass substrate carrier bearing silane after coating five-fold diluted TGF-beta 1 standard by a microwave therapy apparatus. The overall OD values of the glass substrate carrier bearing gold nanoparticles and silane are higher, but after subtracting the background value, the glass substrate carrier bearing gold nanoparticles did not differ significantly from the glass substrate carrier bearing silane. After microwave coating by a microwave therapy apparatus, both of them can achieve good sensitivity.

TABLE 13

| Concen- | The glass substrate carrier | | The glass substrate | |
|---|---|---|---|---|
| tration | bearing gold nanoparticles and | | carrier bearing silane | |
| (pg/ml) | silane of the present invention | | of the present invention | |
| 1000 | 3.860 | Exceed the | 3.923 | 3.818 |
| | | measurable | | |
| | | limit | | |
| 200 | 1.256 | 1.157 | 1.379 | 1.266 |
| 40 | 0.3055 | 0.2884 | 0.3076 | 0.3053 |
| 4 | 0.1175 | 0.1037 | 0.08846 | 0.07630 |
| 0.8 | 0.07916 | 0.06957 | 0.03338 | 0.03237 |
| 0.16 | 0.07154 | 0.06279 | 0.02192 | 0.02053 |
| 0.032 | 0.06608 | 0.05737 | 0.01938 | 0.01950 |
| Blank | 0.06849 | 0.06165 | 0.02013 | 0.02013 |
| control | | | | |
| group | | | | |

### EMBODIMENTS

1. A method for manufacturing a measuring kit, comprising steps of: providing a carrier including a substrate having a surface; coating a silane onto the surface of the substrate; providing an organic molecule; adding the organic molecule onto the carrier; outputting a microwave energy by a coaxial cable; and emitting the microwave energy through a radiator to microwave the carrier and the organic molecule uniformly to coat the organic molecule onto the carrier carrying the silane, wherein the measuring kit is used to apply an enzyme-linked immunosorbent assay.
2. The method of Embodiment 1, wherein the microwaving step is performed under 60 W for 30-40 minutes.
3. The method of any one of Embodiments 1-2, wherein the carrier bears a plurality of spaced metal nanoparticles.
4. The method of any one of Embodiments 1-3, wherein the carrier is one selected from a group consisting of a protein chip, a gene chip and a microchannel chip.
5. A method for manufacturing a measuring kit, comprising steps of: providing a carrier including a substrate having a surface; coating a silane onto the surface of the substrate; providing an organic molecule; adding the organic molecule onto the carrier; outputting a microwave energy by a coaxial cable; and emitting the microwave energy through a radiator uniformly to microwave the carrier and the organic molecule to coat the organic molecule onto the carrier carrying the silane.
6. The method of Embodiment 5, wherein the microwaving step is performed under 60 W for 30-40 minutes.
7. The method of any one of Embodiments 5-6, wherein the microwaving step is performed under 60 W for 30 minutes.
8. The method of any one of Embodiments 5-7, wherein the carrier bears a plurality of spaced metal nanoparticles.
9. The method of any one of Embodiments 5-8, wherein the carrier is one selected from a group consisting of a protein chip, a gene chip and a microchannel chip.
10. The method of any one of Embodiments 5-9, wherein the substrate is made of one of a glass and a plastic.
11. The method of any one of Embodiments 5-10, wherein the measuring kit is applied to a measuring method being one selected from a group consisting of a sandwich enzyme-linked immunosorbent assay, an indirect enzyme-linked immunosorbent assay and a competitive enzyme-linked immunosorbent assay.
12. A method for accelerating an attachment of an organic molecule to a carrier, comprising steps of: providing the carrier including a substrate having a surface; coating a silane onto the surface of the substrate; adding the organic molecule onto the carrier; and increasing a collision probability between the carrier carrying the silane and the organic molecule with a specific power to accelerate the attachment of the organic molecule to the carrier carrying the silane.
13. The method of Embodiment 12, wherein the specific power is provided when a microwave energy is outputted by a coaxial cable and is uniformly emitted through a radiator to microwave the carrier carrying the silane and the organic molecule.
14. The method of any one of Embodiments 12-13, wherein the organic molecule is one of an antibody and an antigen.
15. The method of any one of Embodiments 12-14, wherein the specific power has a fixed value.
16. The method of any one of Embodiments 12-15, wherein the carrier carrying the silane and the organic molecule are microwaved by a power less than 200 W.
17. The method of any one of Embodiments 12-16, wherein the carrier carrying the silane and the organic molecule are microwaved by a power of 60 W for 30-40 minutes.
18. The method of any one of Embodiments 12-17, wherein the carrier bears a plurality of spaced metal nanoparticles.
19. The method of any one of Embodiments 12-18, wherein the carrier is one selected from a group consisting of a protein chip, a gene chip and a microchannel chip.
20. The method of any one of Embodiments 12-19, wherein the substrate is made of one of a glass and a plastic.

While the invention has been described in terms of what is presently considered to be the most practical and preferred embodiments, it is to be understood that the invention need not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method for manufacturing a measuring kit, comprising steps of:
providing a carrier including a substrate having a surface;
coating a silane onto the surface of the substrate;
providing an organic molecule;
adding the organic molecule onto the carrier;
outputting a microwave energy by a coaxial cable; and
emitting the microwave energy through a radiator to microwave the carrier and the organic molecule uniformly to coat the organic molecule onto the carrier carrying the silane, wherein the measuring kit is used to apply an enzyme-linked immunosorbent assay.

2. The method according to claim 1, wherein the microwaving step is performed under 60 W for 30-40 minutes.

3. The method according to claim 1, wherein the carrier bears a plurality of spaced metal nanoparticles.

4. The method according to claim 1, wherein the carrier is one selected from a group consisting of a protein chip, a gene chip and a microchannel chip.

5. A method for manufacturing a measuring kit, comprising steps of:
providing a carrier including a substrate having a surface;
coating a silane onto the surface of the substrate;
providing an organic molecule;
adding the organic molecule onto the carrier;
outputting a microwave energy by a coaxial cable; and
emitting the microwave energy through a radiator uniformly to microwave the carrier and the organic molecule to coat the organic molecule onto the carrier carrying the silane.

6. The method according to claim 5, wherein the microwaving step is performed under 60 W for 30-40 minutes.

7. The method according to claim 5, wherein the microwaving step is performed under 60 W for 30 minutes.

8. The method according to claim 5, wherein the carrier bears a plurality of spaced metal nanoparticles.

9. The method according to claim 5, wherein the carrier is one selected from a group consisting of a protein chip, a gene chip and a microchannel chip.

10. The method according to claim 5, wherein the substrate is made of one of a glass and a plastic.

11. The method according to claim 5, wherein the measuring kit is applied to a measuring method being one selected from a group consisting of a sandwich enzyme-linked immunosorbent assay, an indirect enzyme-linked immunosorbent assay and a competitive enzyme-linked immunosorbent assay.

12. A method for accelerating an attachment of an organic molecule to a carrier, comprising steps of:
providing the carrier including a substrate having a surface;
coating a silane onto the surface of the substrate;
adding the organic molecule onto the carrier; and
increasing a collision probability between the carrier carrying the silane and the organic molecule with a specific power to accelerate the attachment of the organic molecule to the carrier carrying the silane.

13. The method according to claim 12, wherein the specific power is provided when a microwave energy is outputted by a coaxial cable and is uniformly emitted through a radiator to microwave the carrier carrying the silane and the organic molecule.

14. The method according to claim 12, wherein the organic molecule is one of an antibody and an antigen.

15. The method according to claim 12, wherein the specific power has a fixed value.

16. The method according to claim 15, wherein the carrier carrying the silane and the organic molecule are microwaved by a power less than 200 W.

17. The method according to claim 16, wherein the carrier carrying the silane and the organic molecule are microwaved by a power of 60 W for 30-40 minutes.

18. The method according to claim 12, wherein the carrier bears a plurality of spaced metal nanoparticles.

19. The method according to claim 12, wherein the carrier is one selected from a group consisting of a protein chip, a gene chip and a microchannel chip.

20. The method according to claim 12, wherein the substrate is made of one of a glass and a plastic.
